(19) United States
(12) Patent Application Publication
(10) Pub. No.: US 2015/0305929 A1
(43) Pub. Date: Oct. 29, 2015

(54) MAGNETIC CONTACT LENSES AND METHODS OF TREATMENT AND DIAGNOSIS USING THE SAME

(71) Applicant: EMMETROPE OPHTHALMICS LLC, Key Biscayne, FL (US)

(72) Inventors: Jeffrey L. Goldberg, San Diego, CA (US); Roger A. Goldberg, Boston, MA (US); Noelia J. Kunzevitzky, Miami, FL (US); Stavros Nickolas Moysidis, Miami, FL (US)

(73) Assignee: EMMETROPE OPHTHALMICS LLC, Key Biscayne, FL (US)

(21) Appl. No.: 14/441,466

(22) PCT Filed: Nov. 5, 2013
(86) PCT No.: PCT/US13/68401

§ 371 Date: May 7, 2015

Related U.S. Application Data
(60) Provisional application No. 61/723,480, filed on Nov. 7, 2012.

(51) Int. Cl. A61F 9/00; G02C 7/04

(52) U.S. Cl. CPC A61F 9/0017; G02C 7/04; A61F 2210/009; A61F 9/0026

(57) ABSTRACT

In certain aspects, the invention is directed to magnetic contact lenses that comprise one or more magnets. When worn by a patient, the magnetic contact lenses are configured to generate an intraocular magnetic field of sufficient magnitude and direction to move a magnetic therapeutic and/or diagnostic agent positioned inside the eye to target tissue within the eye. Other aspects of the invention pertain to kits which comprise such magnetic contact lenses as well as one or more additional components, for example, one or more containers of a magnetic diagnostic and/or or therapeutic agent. Further aspects of the invention pertain to methods of treatment, which comprise intraocularly introducing a magnetic therapeutic and/or diagnostic agent into an eye of a patient and fitting a magnetic contact lens to the head of the patient, wherein the magnetic therapeutic and/or diagnostic agent may be introduced to the patient before or after fitting the magnetic contact lens to the head of the patient.

### STATEMENT OF RELATED APPLICATION

This application claims the benefit of U.S. Ser. No. 61/723,480, filed Nov. 7, 2012 and entitled: “MAGNETIC CONTACT LENSES AND METHODS OF TREATMENT AND DIAGNOSIS USING THE SAME,” which is hereby incorporated by reference in its entirety.

### FIELD OF THE INVENTION

The present invention is related to magnetic contact lenses and to methods of treatment and diagnosis using magnetic contact lenses.

### BACKGROUND

A large number of diseases and disorders result from the dysfunction of a specific tissue or organ. A number of these diseases and disorders are currently treated by transplantation, e.g., heart transplantation for certain types of cardiac dysfunction, corneal transplantation for corneal endothelial cell dysfunction, stem cells for blood cancers, and so forth. However, transplantation procedures are invasive, have varying rates of success, and are not available for many types of injuries, diseases or disorders, in particular for a number of eye diseases, for example, including certain injuries or diseases of the cornea (e.g., endothelial dystrophies, stromal dystrophies, bullous keratopathy, etc.), certain injuries or diseases of retinal ganglion cells and the optic nerve (e.g., glaucoma, retinal artery or vein occlusions, ischemic optic neuropathies, other optic neuropathies, etc.), and certain diseases of retinal photoreceptors and retinal pigment epithelium (e.g., Leber's congenital amaurosis, retinitis pigmentosa, age-related macular degeneration, etc.) For ease of reference, various parts of the eye 10 are shown in FIG. 1, specifically, the cornea 1, pupil 2, iris 3, ciliary muscle 6, lens 4, retina 5, optic nerve 7 and anterior chamber 8 (which contains the aqueous humor), and vitreous cavity 9.

Although in many cases it would seem desirable to administer new “healthy” cells, for instance, by injection or infusion, simply introducing such cells into the eye generally does not work as they do not remain localized and adhere to or become incorporated into the target tissue of a patient. For example, healthy corneal endothelial cells are inefficiently incorporated into a patient's diseased or injured cornea when injected into the anterior chamber of the eye, with the majority of cells simply falling by gravity away from the cornea, rather than properly attaching to the cornea (see, e.g., Mimura et al., Invest. Ophthalmol. Vis. Sci. 2005, 46(10):3637-44). Similarly, healthy retinal ganglion cells are not incorporated into the retina when injected into the vitreous cavity of the eye (see, e.g., U.S. 2011/0003003 to Goldberg et al., the disclosure of which is hereby incorporated by reference).

### SUMMARY OF THE INVENTION

In certain aspects, the invention is directed to magnetic contact lenses that comprise a magnet. When worn by a patient, the magnetic contact lenses are configured to generate an intraocular magnetic field of sufficient magnitude and direction to move a magnetic therapeutic and/or diagnostic agent positioned inside the eye to a target tissue within the eye.

Other aspects of the invention pertain to kits which comprise such magnetic contact lenses as well as one or more additional components, for example, one or more containers of a magnetic diagnostic and/or or therapeutic agent.

Further aspects of the invention pertain to methods of treatment, which comprise intraocularly introducing a magnetic therapeutic and/or diagnostic agent into an eye of a patient and fitting a magnetic contact lens to the eye of the patient. The magnetic contact lens is configured to generate an intraocular magnetic field of sufficient magnitude and direction to move the magnetic therapeutic and/or diagnostic agent positioned inside the eye to a target tissue within the eye, and the magnetic therapeutic and/or diagnostic agent may be introduced to the patient before or after fitting the magnetic contact lens.

These and various other aspects and embodiments and as well as advantages of the present invention will become immediately apparent to those of ordinary skill in the art upon review of the Detailed Description and any appended claims to follow.

### BRIEF DESCRIPTION OF THE DRAWINGS

FIG. 1 is a schematic illustration of a human eye in accordance with the prior art.

FIG. 2A is a schematic illustration of a bar magnet and associated field lines, in accordance with the prior art.

FIG. 2B is a schematic illustration of a ring-shaped magnet and associated field lines, in accordance with the prior art.

FIG. 3 is a schematic illustration of a contact lens with an associated ring-shaped magnet like that of FIG. 2B, in accordance with an embodiment of the present invention.

FIG. 4 is a schematic illustration showing a contact lens like that of FIG. 3 in contact with the eye, in accordance with an embodiment of the present invention.

FIG. 5A is a schematic illustration showing a contact lens with an associated magnet, in accordance with an embodiment of the present invention.

FIG. 5B is a schematic illustration showing a contact lens with an associated electromagnet, in accordance with another embodiment of the present invention.

FIG. 6 is a schematic illustration of a contact lens with an associated ring-shaped magnet like that of FIG. 2B, in accordance with another embodiment of the present invention.

FIG. 7 is a schematic illustration of a cross-section of a bar magnet and associated field lines, for use in various embodiments of the invention.

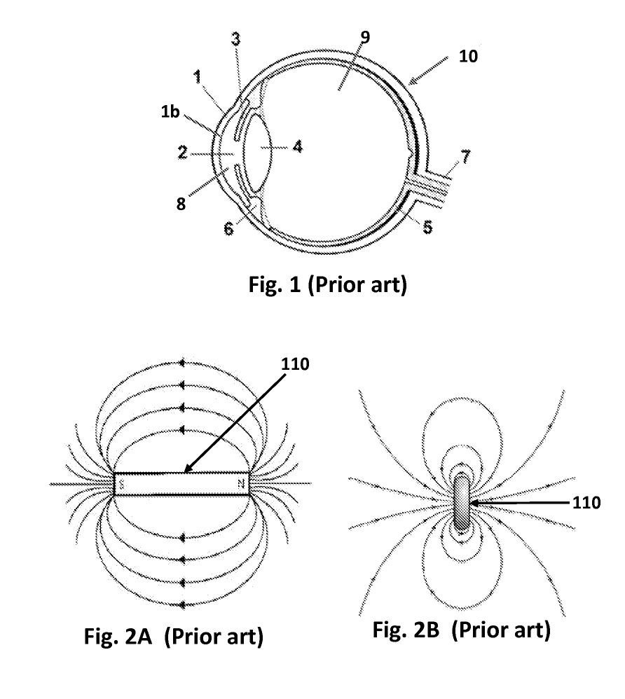

### DETAILED DESCRIPTION OF THE INVENTION

A more complete understanding of the present invention is available by reference to the following detailed description of numerous aspects and embodiments of the invention. The detailed description of the invention which follows is intended to illustrate but not limit the invention.

In the present disclosure, magnetic contact lenses are provided which are adapted to preferentially position magnetic diagnostic and/or or therapeutic agents which are placed within the eye of a subject for a variety of purposes. Though there are many contact lenses for novelty, corrective or protective purposes, no contact lens is known to be available that by design provides a specifically desired intraocular magnetic field for the preferentially positioning magnetic diagnostic and/or or therapeutic agents.

As used herein, “subjects” (also referred to as “patients”) are vertebrate subjects, more typically mammalian subjects, including human subjects, pets and livestock.

Most materials can be classified as diamagnetic, paramagnetic, ferromagnetic or ferrimagnetic. Diamagnetic materials have a weak, negative susceptibility to magnetic fields and are thus slightly repelled by a magnetic field. Most elements in the periodic table, including copper, silver, and gold, are diamagnetic. Paramagnetic materials have a small, positive susceptibility to magnetic fields and are thus slightly attracted by a magnetic field. Paramagnetic materials include magnesium, molybdenum, lithium, and tantalum. Ferromagnetic and ferrimagnetic materials have a large, positive susceptibility to an external magnetic field and thus are strongly attracted by a magnetic field. Examples of ferromagnetic materials include iron, nickel, cobalt and some rare earth elements (e.g., gadolinium, dysprosium, etc.). Examples of ferrimagnetic materials include magnetite, maghemite and various ferrites including nickel ferrite, cobalt ferrite, manganese ferrite, nickel zinc ferrite and manganese zinc ferrite. Superparamagnetism is a form of magnetism, which appears in small ferromagnetic or ferrimagnetic nanoparticles (e.g., small particles ranging from 1-25 nm in diameter, more typically, 1-10 nm in diameter). Superparamagnetic materials are attracted by a magnetic field but relax their magnetic dipole when the field is removed, decreasing their ability to attract each other in the absence of an external magnetic field. For diagnostic and therapeutic use, this relaxation may provide certain advantages, in some embodiments.

In the present disclosure, magnetic diagnostic and/or or therapeutic agents are preferably ferromagnetic or ferrimagnetic in nature, and more preferably superparamagnetic in certain applications. Specific examples of therapeutic agents include magnetic cells, for examples magnetic stem cells or magnetic ocular cells such as magnetic corneal endothelial cells and magnetic retinal pigment epithelial cells or magnetic photoreceptor cells. Further specific examples of magnetic therapeutic agents include magnetic growth factors, small molecule drugs, biological therapeutics, antibodies or antibody fragments, or cytokines. Specific examples of diagnostic agents include diagnostic agents such as magnetic fluorescent dyes, magnetic antibodies or antibody fragments, or magnetic particles that could be paired with diagnostic imaging or sensing devices such as optical coherence tomography, ultrasound, and photographic filters. Various materials can be rendered ferromagnetic or ferrimagnetic by associating them with ferromagnetic or ferrimagnetic particles such as microparticles or nanoparticles. For instance, (a) the agents can be attached to the surface of the particles by covalent interactions and/or non-covalent interactions (e.g., interactions such as van der Waals forces, hydrophobic interactions and/or electrostatic interactions, for instance, charge-charge interactions, charge-dipole interactions, and dipole-dipole interactions, including hydrogen bonding), (b) the agents can be applied as a coating (biostable or biodegradable) that at least partially surrounds the particles, or (c) the particles can be bound to or endocytosed by the agent (e.g., a cell) and in either or both cases incorporated into the inside of the agent.

The contact lenses of the present disclosure may be transparent (i.e., having a transmission of visible light of at least 20%), opaque or a combination of both. For example, in certain embodiments, the contact lens of the present disclosure may be transparent, in which case the contact lens may or may not provide vision correction for one or both eyes.

The contact lenses of the present disclosure may comprise a soft, flexible material or may comprise a rigid, preferably gas permeable, material. Examples of soft flexible materials may be formed using one or more polymers such as combination of polymers such as silicones, silicone hydrogels or other hydrogel materials (e.g., materials containing homopolymers or copolymers of two or more hydrogel monomers, such as 2-hydroxyethyl methacrylate, 1-vinyl-2-pyrrolidone, methacrylic acid, etc.). Examples of rigid materials include silicone acrylate (S/A) copolymers, fluorosilicone acrylate (F-S/A) copolymers, and poly(methyl methacrylate) (PMMA). In either case, the magnet may be embedded in the lens or affixed to the surface of the lens.

The lenses preferably have a curvature so as to match the natural curvature of the cornea and provide a standard fit. In the case of a soft contact lens, this may be one size. For example, the contact lenses may be provided in a range of standard corneal curvatures (e.g., a base curve ranging 8 mm to 10 mm, among other values) and may have a range of diameters (e.g., a diameter ranging from 8 mm to 18 mm, among other values, to provide a comfortable and safe fit for the patient. An ideal size for a soft contact lens may be a base curve of 8.8 mm and a diameter of 14 mm, among other values.

In order to generate a magnetic field having a desired magnitude and direction, the magnetic contact lenses of the present disclosure are provided with one or more suitable magnets which may be selected, for example, from temporary magnets, permanent magnets and electromagnets.

Examples of permanent and temporary magnets include magnets that comprise iron, magnets that comprise neodymium, magnets that comprise cobalt, and magnets that comprise boron. Specific examples include rare earth magnets such as magnets that comprise neodymium, iron and boron (e.g., neodymium-iron-boron magnets, which commonly contain an alloy of neodymium, iron and boron, commonly in the form of a Nd₂Fe₁₄B tetragonal crystalline structure), magnets that comprise samarium and cobalt (e.g., samarium-cobalt magnets, which are commonly available in two “series”, specifically Series 1:5, which contain one atom of rare earth samarium for every five atoms of cobalt, and Series 2:17, which contain two atoms of rare-earth samarium and 13-17 atoms of transition metals, with the transition metal content being rich in cobalt). Specific examples further include magnets that comprise iron (e.g., ferrite magnets, which commonly have iron (III) oxide as the principle component) and magnets that comprise iron, aluminum, nickel and cobalt (e.g., Alnico magnets, which typically contain 8-12% Al, 15-26% Ni, 5-24% Co, up to 6% Cu, up to 1% Ti, and the balance Fe).

An electromagnet is a type of magnet in which a magnetic field is produced by the flow of electric current, with the strength of magnetic field generated being proportional to the amount of current. The magnetic field disappears when the current is turned off. Typically, electromagnets comprise a conductor (e.g., an insulated wire, a printed or etched conductive line, etc.) in the form of a coil. To increase the magnetic field, a coil with multiple turns may be employed. The magnetic field may be increased by positioning a ferromagnetic material (e.g., iron, etc.) inside the coil to produce a ferromagnetic-core electromagnet.

Where an electromagnet is employed in the contact lenses of the present disclosure, a power source may also be provided in some embodiments. The power source may include, for instance, a non-rechargeable battery or may include a rechargeable battery, which may be recharged, for instance, by connection to an external voltage source via a conductor (e.g., via a wire connection) or by wireless recharging (e.g., by inductive charging in which an alternating electromagnetic field is generated in an external induction coil). In some embodiments, power may be supplied by the patient. For example, power may be provided electromagnetically by the action of the eyelid blinking over the lens. In this regard, a magnetic material (e.g., in the form of a coil or other suitable shape) may be fitted to the eyelid (e.g., by attaching the magnetic material to the eyelid using a suitable adhesive, by suturing, etc.) such that movement of the magnetic material associated with blinking of the eyelid will induce a voltage inside of a coil implanted in the contact lens.

The power source may also include components which control the current within the electromagnet (and thus the field strength of the electromagnet) and which control the duty cycle of the electromagnet, in other words that amount of time and frequency the electromagnet is “on” (and generating a magnetic field) and when it is “off” (and not generating a field). One advantage of the use of an electromagnet in this embodiment is the ability to titrate the field strength exerted by the magnetic contact lens invention by changing the input current to the electromagnet.

For purposes of illustration, two magnets and their associated magnetic field lines are shown schematically in FIGS. 2A and 2B.

FIG. 2A is a schematic illustration of a simple bar magnet 110 (e.g., a rare earth magnet, ferrite magnet, Alnico magnet, etc.) and the magnetic field lines associated with that magnet.

FIG. 2B is a schematic illustration of a ring-shaped magnet 110 and the magnetic field lines associated with the magnet. The ring-shaped magnet 110 may be for example, a temporary or permanent magnet (e.g., a rare earth, ferrite or Alnico magnet with poles on opposing faces of the ring) or the ring-shaped magnet 110 may be an electromagnet.

“Magnetic field lines” are lines that are drawn to show the direction of a magnetic field created by a magnet. These lines are also called “lines of force”. Magnetic materials that are sufficiently mobile will migrate as a result of a magnetic field.

In various aspects, the present disclosure is directed to contact lenses that generate an intraocular magnetic field that is sufficient to physically direct a magnetic therapeutic and/or diagnostic agent (e.g., a ferromagnetic material, ferrimagnetic material, etc.) positioned inside of the eye (e.g., placed in the eye by a patient or health care provider via surface application, infusion, injection, implantation, etc.) to one or more target tissues within the eye.

For instance, in one particular embodiment, the contact lens may generate a magnetic field having a magnitude and direction such that a magnetic diagnostic and/or therapeutic agent positioned in the anterior chamber of the eye is directed to the back surface of the cornea.

In another particular embodiment, the contact lens may generate a magnetic field having a magnitude and direction such that a magnetic diagnostic and/or therapeutic agent positioned in the vitreous cavity of the eye is directed towards the posterior pole of the eye.

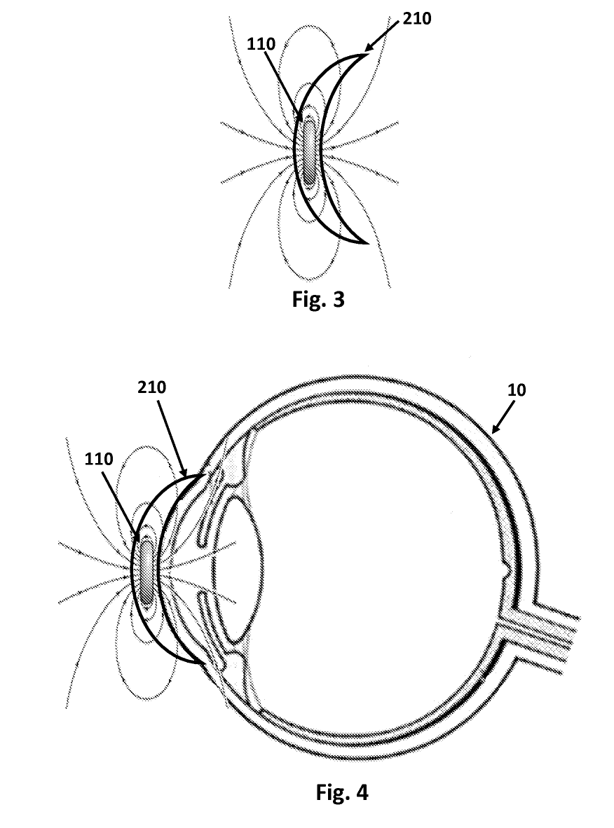

FIG. 3 is a schematic illustration of a contact lens 210 in accordance with the present disclosure within which is disposed a ring-shaped magnet 110 like that of FIG. 2B. As seen schematically in FIG. 4, when a contact lens 210 with magnet 110 like that of FIG. 3 is placed adjacent to an eye 10, the magnetic field lines associated with such a device penetrate the eye, thereby exerting a force on any magnetic material that is disposed within the eye; that force may be attractive or repulsive. Various embodiments of the use of a ring shaped magnet allow a clear center of the magnetic contact lens through which the patient can see along the patient's natural visual axis.

While a ring-shaped magnet like that of FIG. 2B is shown in FIGS. 3 and 4, it should be clear from the present disclosure that the invention is not limited to such a magnet. Other types of magnets may be employed so long as a magnetic field is established within the eye that is capable of directing a magnetic therapeutic and/or diagnostic agent positioned within the eye to a targeted position within the eye.

Different magnetic fields can be used to attract or repel magnetic agents to different locations within the eye. In some embodiments, a magnet placed anterior to the eye will apply an attractive force to a magnetic material (e.g., a paramagnetic, ferromagnetic or ferrimagnetic material) within the eye in a direction that includes an anterior vector component. Consequently, magnets incorporated into external contact lenses in accordance with the present disclosure may be used to draw intraocular magnetized material to the anterior aspect of the eye for diagnostic or therapeutic purposes. In other embodiments, a magnet placed anterior to the eye will apply a repulsive force to a magnetic material (e.g., a diamagnetic material) within the eye in a direction that includes a posterior vector component. Thus, a diamagnetic material may be used which is repulsed from the magnetic field, and, as in the above description, may drive the therapeutic and/or diagnostic agent to the posterior aspect of the eye.

The one or more magnets provided within the contact lenses typically generate a magnetic field strength ranging from 0.01 Tesla or less to 5 Tesla or more (e.g., ranging from 0.01 Tesla to 0.025 Tesla to 0.05 Tesla to 0.1 Tesla to 0.25 Tesla to 0.5 Tesla to 1.0 Tesla to 2.5 Tesla to 5.0 Tesla). More typical magnetic field strengths may range from 0.1 to 1.0 Tesla in order to allow a force sufficient to cover the 24 mm axial length of a typical human eye. The actual field strength will vary depending on various factors including the depth of the target tissue within the eye, and the responsiveness, or magnetic susceptibility, of the therapeutic and/or diagnostic agent to the magnetic field, among other factors.

In some embodiments, the contact lenses of the present disclosure are configured to provide a magnetic field of constant field strength in time. In other embodiments, the contact lenses of the present disclosure are configured to provide a magnetic field of variable field strength as function of time. For example, it may be advantageous to be able to create a magnetic field that has an on/off duty cycle to control the extent and duration of the magnetic field, or to reverse the polarity. This may be able to help a magnetic agent placed inside the eye to circulate for extended periods of time within the eye. In another example, it may be advantageous to vibrate the magnetic agent inside the eye to generate mechanical forces or heat. In another example, it may be advantageous to titrate a magnetic field strength up or down to maximize the proposed delivery of the magnetic diagnostic and/or therapeutic device adjacent to or inside the eye.

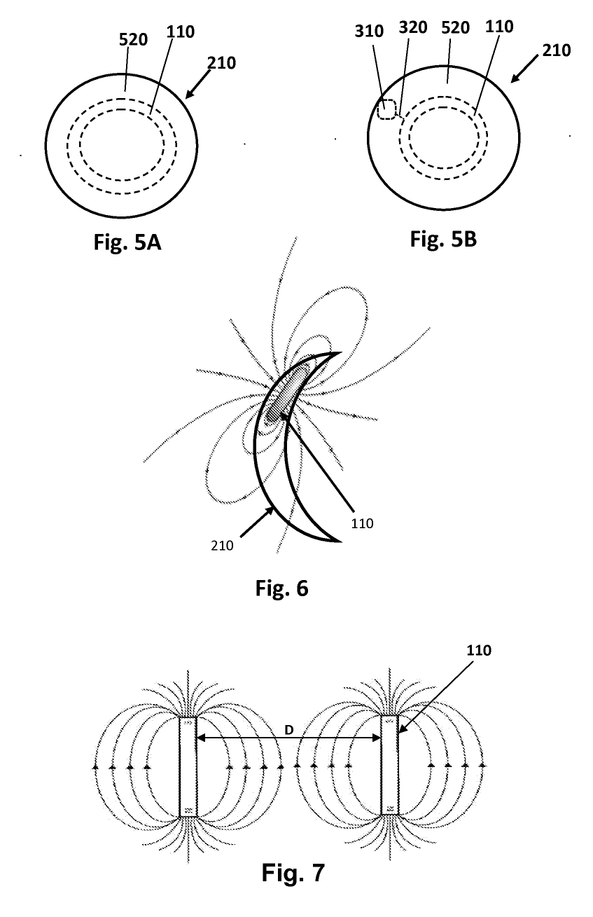

Turning now to FIG. 5A, a magnetic contact lens 210 in accordance with an embodiment of the present disclosure is schematically shown. The magnetic contact lens 210 shown includes a lens portion 510, which may be corrective or non-corrective. For example, lens portion correspond to a soft contact lens with the base curve of 8.8 mm and a diameter of 14 mm, among other possibilities. The magnetic contact lens 210 also includes a magnet 110, specifically a ring shaped magnet which may be, for example, a ferrite magnet, an Alnico magnet, or more preferably, a rare earth magnet such as a neodymium-iron-boron magnet or a samarium-cobalt magnet, among other possibilities. The magnet may range, for example, from 4 mm to 18 mm in diameter, more commonly from 8 to 12 mm in diameter, and may range from 0.01 to 6.0 Tesla in field strength, more commonly between 0.1 and 1.0 Tesla in field strength. The magnet may be attached to the surface of the lens (e.g., using a suitable adhesive) or may be embedded in the lens. In many embodiments, the magnetic field is centered on the optical axis of the eye. In other embodiments, the magnetic field is not centered on the optical axis of the eye.

Turning now to FIG. 5B, a magnetic contact lens 210 in accordance with another embodiment of the present disclosure is schematically shown. The magnetic contact lens 210 shown includes a lens portion 510, which may be corrective or non-corrective. For example, lens portion correspond to a soft contact lens with the base curve of 8.8 mm and a diameter of 14 mm, among other possibilities. The magnetic contact lens 210 also includes an electromagnet 110 which comprises a conductive coil having one or more loops, which may be formed using lines of a transparent conductor (e.g., formed of indium tin oxide, fluorine doped tin oxide, doped zinc oxide, etc.) or using an opaque conductor (e.g., a metallic conductor such as copper, silver, gold, aluminum, etc.). The magnet may range, for example, from 4 mm to 18 mm in diameter, more commonly from 8 to 12 mm in diameter, and may range from 0.01 to 6.0 Tesla in field strength, more commonly between 0.1 and 1.0 Tesla in field strength. The magnet may be attached to the surface of the lens (e.g., using a suitable adhesive) or may be embedded in the lens. In many embodiments, the magnetic field is centered on the optical axis of the eye. In other embodiments, the magnetic field is not centered on the optical axis of the eye.

In some embodiments, the magnetic contact lens 210 further includes a power supply 310 like that described above, which is connected to the electromagnet 110 via one or more conductive lines 320 which, like the coils of the electromagnet 110, may be formed using a transparent conductor or an opaque conductor.

As previously indicated, in certain preferred embodiments, contact lenses of the present disclosure are provided with magnetic fields that are centered with the optical axis of the eye.

In some of these embodiments (see, e.g., the schematic illustration in FIG. 4) the magnetic field directs magnetic therapeutic and/or diagnostic agents which have been positioned within the eye toward the optical axis of the eye. Because the magnet is disposed anterior to the eye, the magnetic field for such devices will be the strongest at the apex of the cornea. Consequently, magnetic therapeutic and/or diagnostic agents placed in the anterior chamber of the eye can be directed to the center of the cornea along the endothelial surface. In certain embodiments, this will help prevent the magnetic therapeutic and/or diagnostic agents from settling into the inferior anterior chamber where the cells may clog the trabecular meshwork and limit aqueous egress from the eye. Additionally, this will direct the material into the optical axis where a therapeutic and/or diagnostic effect is desired.

In other embodiments, magnetic contact lenses may be configured to generate an intraocular magnetic field that is strongest in a position other than the corneal apex. For example, the magnetic contact lens may be configured to generate an intraocular magnetic field that is strongest at the periphery of the cornea, for example, at the iridocorneal angle (where the base of the iris attaches to the peripheral cornea and sclera), among other locations. These embodiments may be useful, for example, in treatment of glaucoma using trabecular meshwork cells, among other treatments.

In some embodiments, a magnet having a magnetic field like that of FIG. 2B may be used to form a magnetic contact lens in which the magnet and its associated magnetic field are off-center with respect to the center of the contact lens. One example of a contact lens 210 with such an off-center magnet 110 is shown schematically in FIG. 6. In some embodiments the lens 210 may be weighted to position the center of the magnet 110 at a particular rotational position.

In other embodiments, a magnet may be employed which is on-center with the regard to the contact lens and which nevertheless does not generate an intraocular magnetic field that is strongest at the corneal apex. For example, a ring-shaped magnet 110 like that shown in schematic cross-section in FIG. 7 (where D is the inside diameter of the magnet) may be employed, in which one surface represents a north pole of the magnet and another opposing surface represents a south pole of the magnet. Such a magnet is capable of providing a circular region of maximum intraocular field strength whose diameter can be adjusted based on the diameter of the magnet. For example, the diameter of the magnet may be adjusted to provide a maximum intraocular field strength at the periphery of the cornea, as indicated above. In this instance, the center of the magnet is preferably centered with respect to the contact lens. In other embodiments, the center of such a magnet may be off-center with regard to the contact lens.

Further aspects of the present disclosure pertain to methods of treatment of a subject.

In a typical procedure, a magnetic therapeutic and/or diagnostic agent is introduced into the eye, for example, by injection, implantation, infusion, or surface application, among other techniques. Injection or implantation may be preferred in certain embodiments as more control is provided other than placement of the material within the eye which, in turn, assists in directing the agent to target tissue of choice. A magnetic contact lens such as one of those described elsewhere herein is also fitted to the eye of the subject, either prior or subsequent to the introduction of the magnetic therapeutic and/or diagnostic agent.

The magnetic contact lens is left in position for a time that is dependent upon various factors including the type of magnetic therapeutic and/or diagnostic agent employed and the length of time required to see a clinical effect, whether for therapeutic or diagnostic purposes. The time frame may varying anywhere from 10 minutes to indefinitely. Typical time frames may range, for example, from 3 hours to 72 hours, among others.

In one particular embodiment, a procedure is provided in which a magnetic therapeutic and/or diagnostic agent is introduced into the anterior chamber, and a magnetic contact lens may be worn to draw those materials anteriorly to the apical aspect of the corneal endothelium. For example, magnetic corneal endothelial cells can be injected in to the anterior chamber of one or both eyes and one or a pair of magnetic contact lenses can be worn for anywhere from 10 minutes to indefinitely but typically for 1-3 days after injection to stimulate migration of the injected corneal endothelial cells to the back surface of the cornea to facilitate integration and retention of these cells into the host corneal endothelium.

Still further aspects of the present disclosure pertain to kits that are useful for diagnosing or treating a patient. The kits may include all or a subset of all the components useful for treating or diagnosing a patient in accordance with the present disclosure. The kits may include, for example, any combination of two or more of the following items: (a) one or more magnetic contact lenses in accordance with the present disclosure, (b) one or more containers of a magnetic diagnostic and/or or therapeutic agent, for example, in a form that is suitable for immediate administration to a patient (e.g., in a liquid form suitable for injection, infusion or surface application, in a dry form suitable for implantation, etc.) or in a form suitable for administration upon addition of another component (e.g., in a dry form that is suitable for administration upon suspension or dissolution using a suitable liquid carrier, (c) one or more containers of a suitable liquid carrier (e.g. sterile water for injection, physiological saline, phosphate buffer, phosphate buffered saline, etc.) which may be used to reconstitute a magnetic diagnostic and/or or therapeutic agent in dry form or may be used to dilute a magnetic diagnostic and/or or therapeutic agent in liquid form, (d) an injection device (e.g., a combination syringe and needle or an iontophoresis device for administering a composition comprising a magnetic diagnostic and/or or therapeutic agent to the patient's eye), (e) instructions for administering the magnetic compositions to a patient's eye and/or for fitting the magnetic contact lens, (f) packaging and information as required by a governmental regulatory agency that regulates cell therapy products, pharmaceuticals and/or medical devices, and (g) appropriate anesthetic and antiseptic supplies

In certain embodiments, the components of the kits are provided in a single sterile package for convenient use by a health care professional.

Where the kit comprises a magnet that is not an electromagnet (e.g., a rare earth magnet, ferrite magnet, Alnico magnet, etc.) in combination with a ferromagnetic or ferromagnetic diagnostic and/or therapeutic agent, it may be desirable to provide the kit with shielding to magnetically isolate the ferromagnetic or ferromagnetic agent from the magnet. For instance, if exposed to a magnetic field of sufficient magnitude for a sufficient time, the ferromagnetic or ferromagnetic diagnostic and/or therapeutic agent may itself become magnetized, which may lead, for example to clumping of the agent. In embodiments where shielding is desired, the ferromagnetic or ferromagnetic diagnostic and/or therapeutic agent, the magnet, or both may be enclosed within a suitable magnetic shielding material. Examples of magnetic shielding materials include various high-permeability shielding alloys such as nickel-iron alloys including permalloy (an alloy of nickel and iron) and mu-metal (an alloy of nickel, iron, copper and molybdenum or chromium), among others.

Although various embodiments are specifically illustrated and described herein, it will be appreciated that modifications and variations of the present invention are covered by the above teachings and are within the purview of any appended claims without departing from the spirit and intended scope of the invention.

1. A magnetic contact lens comprising a magnet that when worn by a patient is configured to generate an intraocular magnetic field of sufficient magnitude and direction to move a magnetic therapeutic and/or diagnostic agent positioned inside the eye to a target tissue within the eye.

2. The magnetic contact lens of claim 1, wherein the magnetic field generated by the magnet ranges from 0.1 Tesla to 10.0 Tesla.

3. The magnetic contact lens of claim 1, wherein the magnet is a rare earth magnet.

4. The magnetic contact lens of claim 1, wherein the magnet is an electromagnet comprising a conductive coil and a source of electrical power in electrical communication with the conductive coil.

5. The magnetic contact lens of claim 1, wherein the magnet is an electromagnet comprising a conductive coil and wherein electrical power is derived from an external inductive charging coil or from blinking of an eyelid of the patent to which a magnet is attached.

6. The magnetic contact lens of claim 1, wherein the magnetic contact lens is configured to center the magnetic field of the magnet with the optical axis of the eye.

7. The magnetic contact lens of claim 1, wherein the magnetic contact lens is configured to generate an intraocular magnetic field that is strongest at the apex of the cornea.

8. The magnetic contact lens of claim 1, wherein the magnetic contact lens is configured to generate an intraocular magnetic field that is strongest at the periphery of the cornea.

9. The magnetic contact lens of claim 1, wherein the magnetic contact lens is a rigid, gas permeable lens.

10. The magnetic contact lens of claim 1, wherein the magnetic contact lens is a soft lens.

11. The magnetic contact lens of claim 1, wherein the magnetic therapeutic and/or diagnostic agent is a ferromagnetic or ferrimagnetic therapeutic and/or diagnostic agent.

12. A kit comprising a magnetic contact lens in accordance with claim 1 and a container of a magnetic diagnostic and/or or therapeutic agent.

13. The kit of claim 12, further comprising an injection device.

14. The kit of claim 12, wherein the magnetic therapeutic and/or diagnostic agent is a ferromagnetic or ferrimagnetic therapeutic and/or diagnostic agent.

15. The kit of claim 12, wherein the magnetic therapeutic and/or diagnostic agent is selected from one or more of magnetic stem cells, magnetic corneal endothelial cells, magnetic retinal pigment epithelial cells, magnetic trabecular meshwork cells, magnetic antibodies, magnetic growth factors, and magnetic cytokines.

16. The kit of claim 12, wherein the magnet is an electromagnet comprising a conductive coil and wherein the kit further comprises an inductive charging unit or an additional magnet which is securable to an eyelid of the patient and is configured to power the electromagnet by blinking of the eyelid.

17. A method of treatment comprising intraocularly introducing a magnetic therapeutic and/or diagnostic agent into an eye of a patient and fitting a magnetic contact lens to the eye of the patient, wherein the magnetic contact lens is configured to generate an intraocular magnetic field of sufficient magnitude and direction to move the magnetic therapeutic and/or diagnostic agent positioned inside the eye to a target tissue within the eye and wherein the magnetic therapeutic and/or diagnostic agent may be introduced to the patient before or after fitting the magnetic contact lens to the head of the patient.

18. The method of claim 17, wherein the magnetic therapeutic and/or diagnostic agent is injected into the anterior chamber of the eye and wherein the magnetic therapeutic and/or diagnostic agent is directed to the apex of the cornea.

19. The method of claim 17, wherein the magnetic therapeutic and/or diagnostic agent is injected into the anterior chamber of the eye and wherein the magnetic therapeutic and/or diagnostic agent is directed to the periphery of the cornea.

20. The method of claim 17, wherein the magnetic therapeutic and/or diagnostic agent is selected from one or more of magnetic stem cells, magnetic corneal endothelial cells, magnetic retinal pigment epithelial cells, magnetic trabecular meshwork cells, magnetic antibodies, magnetic growth factors, and magnetic cytokines.

21. The method of claim 17, wherein the magnetic therapeutic and/or diagnostic agent is selected from magnetic drugs and biological therapeutics
